(19) United States
(12) Patent Application Publication
(10) Pub. No.: US 2019/0085304 A1
(43) Pub. Date: Mar. 21, 2019

(54) Method of Preparation of SOD Hydrolysates Powder

(71) Applicant: NE PLUS ENTERPRISES, LLC, Flushing, NY (US)

(72) Inventor: Lijun QING, Shenzhen (CN)

(21) Appl. No.: 15/708,642

(22) Filed: Sep. 19, 2017

(51) Int. Cl. C12N 9/02; C12P 21/06; A61K 9/19; A61K 38/44

(52) U.S. Cl. CPC C12N 9/0089; C12Y 115/01001; C12Y 302/01004; A61K 38/446; C12P 21/06; A61K 9/19; C12Y 304/00

(57) ABSTRACT

A method for preparing powdery superoxide dismutase (SOD) hydrolysates. SOD is hydrolyzed by cellulase and then further hydrolyzed with a solution containing a mixture of proteases. Organic citric acid is added in the SOD hydrolysates solution, then the solution is freeze-dried to obtain the powdery SOD hydrolysates.

### TECHNICAL FIELD

The present invention pertains to methods for preparing superoxide dismutase (SOD) hydrolysates.

### BACKGROUND

Many diseases are caused by oxygen free radicals, such as superoxides. Superoxides are produced as a by-product of oxygen metabolism. SOD can help eliminate harmful superoxides, and thus is an important antioxidant defense in living cells exposed to oxygen. However, commonly available SOD is a macromolecule and cannot be absorbed easily by the digestive tract.

### SUMMARY OF THE INVENTION

The present invention provides a method for preparing powdery SOD hydrolysates. In one embodiment, the method includes:

(a) obtaining a solution containing polypeptides by hydrolyzing an SOD by a cellulase;

(b) obtaining a composite enzyme solution;

(c) mixing the solution containing polypeptides obtained in (a) with the composite enzyme solution obtained in (b) to thereby obtain a solution of SOD hydrolysates;

(d) adding citric acid to the solution of SOD hydrolysates obtained in (c) to thereby form a further mixture; and

(e) freeze-drying the further mixture obtained in (d) to obtain powdery SOD hydrolysates.

In some embodiments, the composite enzyme solution can be obtained by:

A. obtaining a solution containing a protein;

B. adjusting the pH of the solution to about 9.0;

C. adding a basic protease to the solution, heating the solution, cooling the solution, and repeating the heating and cooling until the pH of the solution stabilizes at about 7;

D. adjusting the pH of the solution obtained from step C to about 8.0;

E. adding a neutral protease to the solution, heating the solution, cooling the solution, and repeating the heating and cooling until the pH of the solution stabilizes at about 7;

F. adjusting the pH of the solution obtained from step E to about 3.5;

G. adding an acidic protease to the solution, heating the solution, cooling the solution, and repeating the heating and cooling until the pH of the solution stabilizes at about 2-3.

In some embodiments, the SOD is derived from a plant, such as corn, garlic, onion, wheat, or combinations thereof.

In some embodiments, the protein for making the composite enzyme solution can be a corn protein powder or soybean protein powder.

### DETAILED DESCRIPTION

The purpose of the invention is to provide a method for preparing fine SOD hydrolysate powders, which has very small molecular weight and is easily absorbed by human body.

To accomplish the purpose of the invention, a preparation method for making micronized SOD powders are provided, which comprises the following steps:

Step 1: SOD is pulverized, passed through a 50-100 mesh screen, and dissolved by adding water at a temperature of 40-50° C. and 0.01-0.03 MPa at pH of 6.2-6.5. A cellulase is added to hydrolyze at least some of the SOD at a temperature of 45-55° C. to obtain a solution containing polypeptides. This is saved for later steps.

Step 2: A protein having 10% water content is added a NaOH solution to adjust the pH value to about 9.0. A 6-8% basic protease is added into the mixture, and the mixture is heated in a microwave enzymolysis chamber at a temperature of 60-65° C. The mixture is then removed from the chamber and cooled to 25° C. The above heating and cooling steps are repeated 5-8 times, till the pf1 of the mixture solution stabilizes at about 7.

Step 3: The polypeptides obtained from Step 2 are added a NaOH solution to adjust the pH value to about 8.0. A 6-8% neutral protease is added into the mixture and the mixture is heated in a microwave enzymolysis chamber at a temperature of 45-50° C. The mixture is removed from the chamber and cooled to 25° C. The above heating and cooling steps are repeated 5-10 times, till the pH of the mixture solution stabilizes at about 7.

Step 4: Adjust the pH of the mixture solution obtained by Step 3 to about 3.5, A 6-8% acidic protease is added to this solution, and the mixture is heated in a microwave enzymolysis chamber at a temperature of 45-50° C. The mixture solution is removed from the chamber and cooled to 25° C. The above heating and cooling steps are repeated 6-8 times, till the pH of the mixture solution stabilizes between 2-3, thereby obtaining a composite enzyme solution.

Step 5: The composite enzyme solution obtained by Step 4 is added to the polypeptides solution obtained in Step 1, and the pH of the mixture solution is adjusted to about 4.5. Then the mixture solution is shaken at 45° C. for 2 hours to obtain a solution of SOD hydrolysates.

Step 6: An organic citric acid water solution (e.g., having a concentration of about 5% citric acid) is added into the solution of SOD hydrolysates obtained in Step 5. The mixture is then placed in a rotation reactor at a temperature of 115° C. and a pressure of 0.3 MPa for 3 hours. 3.2% activated carbon is then added to the mixture, which is then kept at 55° C. for 0.5 hour. The mixture solution is filtrated, and the filtrate is freeze dried to obtain powdery SOD hydrolysates.

The SOD hydrolysates obtained from Step 6 can be sent for analysis, e.g., capillary electrophoresis, gel permeation chromatography, or thin layer chromatography.

The hydrolysis time in Step 1 can be about 3-5 hours.

The protein in Step 2 can be soybean protein powder or corn protein powder.

The heating time in the microwave enzymolysis chamber in Step 2 can be about 1-3 min for each heating cycle.

The original SOD used in the methods described herein can be derived from a plant (where the plant source can be corn, garlic, onion, wheat, or any combinations thereof), by the following steps:

a. Washing the plant, adding water and pulverizing the plant to obtain a plant juice;

b. Centrifuging the plant juice obtained from step (a) to obtain a supernatant layer which contains SOD;

c. Condensing the SOD-containing supernatant solution obtained from step (b), to obtain a concentrated SOD solution;

d. Adding 95% ethanol into the concentrated SOD solution obtained from step (c) to purify the solution; and

e. Freeze-drying the solution obtained in step (d) to obtain a macromolecular SOD powder.

The SOD hydrolysates obtained by the present invention can have a molecular weight of about 2,000 to about 10,000 Daltons and can be easily absorbed/digested by humans and animals. Compared to macromolecular SOD enzymes, the SOD hydrolysates are more stable and easier to store. The citric acid can remove the bitter taste of the SOD hydrolysates. The free-drying procedure is advantageous to maintain the activity of the SOD hydrolysates.

It is to be understood that the terms “comprise”, “include” or any other variants thereof are intended to cover non-exclusive inclusion, in such way that the processes, methods, products or equipment comprising a series of elements include not only those elements, but also other elements that are not explicitly listed, or elements that are inherent to such processes, methods, products, or equipment.

The principles and embodiments of the invention have been described by way of examples, and the description of the examples and specific embodiments is merely used to illustrate the core concepts of the invention. Without departing from the principles of the invention, improvements, modifications, or variations can be made or the technical features disclosed can be combined in a manner which is apparent to skilled artisans in the art. These improvements, modifications, variations or combinations are within the scope of the invention.

What is claimed is:

1. A method for preparing powdery SOD hydrolysates, comprising:
(a) obtaining a solution containing polypeptides by hydrolyzing an SOD by a cellulase;
(b) obtaining a composite enzyme solution;
(c) mixing the solution containing polypeptides obtained in (a) with the composite enzyme solution obtained in (b) to thereby obtain a solution of SOD hydrolysates;
(d) adding citric acid to the solution of SOD hydrolysates obtained in (c) to thereby form a further mixture; and
(e) freeze-drying the further mixture obtained in (d) to obtain powdery SOD hydrolysates.

2. The method of claim 1, wherein obtaining the composite enzyme solution comprises:
A. Obtaining a solution containing a protein;
B. adjusting the pH of the solution to about 9.0;
C. adding a basic protease to the solution, heating the solution, cooling the solution, and repeating the heating and cooling until the pH of the solution stabilizes at about 7;
D. adjusting the pH of the solution obtained from step C to about 8.0;
E. adding a neutral protease to the solution, heating the solution, cooling the solution, and repeating the heating and cooling until the pH of the solution stabilizes at about 7;
F. adjusting the of the solution obtained from step E to about 3.5;
G. adding an acidic protease to the solution, heating the solution, cooling the solution, and repeating the heating and cooling until the pH of the solution stabilizes at about 2-3.

3. The method of claim 1, wherein the SOD is derived from a plant.

4. The method of claim 1, wherein the SOD is an SOD derived from one of corn, garlic, onion, wheat, or combinations thereof.

5. The method of claim 2, wherein the protein is selected from soybean protein powder or corn protein powder.
